(19) United States
(12) Patent Application Publication
(10) Pub. No.: US 2026/0102389 A1
(43) Pub. Date: Apr. 16, 2026

(54) METHOD FOR SIMULTANEOUSLY REGULATING CRYSTAL FORMS AND CRYSTAL HABITS OF ARIPIPRAZOLE DRUG AND ARIPIPRAZOLE CRYSTAL FORMS THEREOF

(71) Applicant: GUANGDONG UNIVERSITY OF TECHNOLOGY, GUANGZHOU (CN)

(72) Inventors: Kailin XU, GUANGZHOU (CN); Jierui HUO, GUANGZHOU (CN); Jianping WANG, GUANGZHOU (CN); Jie BAI, GUANGZHOU (CN); Suqing ZHAO, GUANGZHOU (CN)

(21) Appl. No.: 18/954,397

(22) Filed: Nov. 20, 2024

(30) Foreign Application Priority Data
Oct. 14, 2024 (CN) 202411432707X

(51) Int. Cl. A61K 31/496; A61K 9/08; A61K 47/42

(52) U.S. Cl. CPC A61K 31/496; A61K 9/08; A61K 47/42

(57) ABSTRACT

A method for simultaneously regulating crystal forms and crystal habits of aripiprazole drug, including: adding human serum albumin to first solvent to obtain human serum albumin solution; adding the aripiprazole to second solvent to obtain aripiprazole solution; adding the aripiprazole solution to the human serum albumin solution in drops, and cooling to room temperature, to obtain the aripiprazole drug. During the crystallization process, different crystal habits of aripiprazole drug were prepared by adding proteins as an inducer. Two crystal forms of aripiprazole drug, APZ-H1 and APZ-H2, were obtained. The present method creatively applies a human serum albumin to regulate the crystal habits of the aripiprazole drug, replacing other toxic or harmful crystal habit modifiers, thereby ensuring the safety of the drug. This method is simple, efficient, and pollution-free, with no toxicity or harm to humans. It is environmentally friendly and conducive to the realization of green industrial production.

### CROSS REFERENCE TO RELATED APPLICATIONS

This application claims foreign priority of Chinese Patent Application No. 202411432707X, filed on Oct. 14, 2024 in the China National Intellectual Property Administration, the disclosures of all of which are hereby incorporated by reference.

### TECHNICAL FIELD

The present disclosure relates to the technical field of drug preparation, in particular to a method for simultaneously regulating crystal forms and crystal habits of aripiprazole drug and aripiprazole crystal forms thereof.

### BACKGROUND

Crystallization is a key separation and purification technique in the pharmaceutical industry, with over 90% of active pharmaceutical ingredients (APIs) purified through crystallization. Crystallization not only enhances drug purity but also improves solubility, flowability, and stability. For example, poorly soluble drugs can improve water solubility by preparing polymorphs, co-crystals, or salts, or by reducing particle size and adding solubilizers. In addition, crystal morphology (e.g., crystal habit) has a significant impact on the physical and chemical properties of the drugs as well as the formulation process. The diversity in crystal habits (such as needle-like, plate-like, or rod-like forms) usually caused by variations in molecular arrangement, can influence product flowability and particle size distribution, thereby affecting energy consumption and cost in downstream processes.

In the pharmaceutical industry, crystal habit plays an important role in the dissolution rate and stability of drugs. Different crystal habits can lead to changes in the pharmacodynamics and pharmacokinetics of the drug, which in turn affects the drug's efficacy and safety. For example, in pharmaceutical production, the crystal habit of the calcium carbonate (CaCO₃) is an asymmetric needle-like crystals tend to aggregate into cake-like form after standing, while cylindrical crystals do not exhibit this phenomenon. Variations in crystal habit can influence the processing of formulations. For instance, drugs with fine needle-like crystal habits exhibit poor flowability, making them prone to clogging and agglomeration during the formulation process, which may result in difficulties in material handling. Differences in crystal habit also affect tablet compression performance, as the symmetry of the crystal habit is a critical factor in direct compression tablet manufacturing. Additionally, variations in crystal habit can affect the dissolution rate and release profile. For example, in the case of the anticancer drug sorafenib tosylate, the dissolution and absorption profiles in pure water, pH=1.2 gastric fluid simulant, and in vivo in mice show significant differences depending on the crystal morphology, even when the same crystal habit and particle size are used. Therefore, precise control of crystal habit is crucial in the pharmaceutical industry. By regulating the crystal habit of a drug, its performance can be optimized, and the subsequent drug manufacturing process can be made more efficient.

Crystal habit is effected by various parameters, including growth rate, intermolecular forces, solvent choice, supersaturation, temperature conditions, stirring intensity, and the presence of additives. Controlling crystal habit involves promoting the stable growth of low-energy crystal faces by reducing the growth rate of those faces, while increasing supersaturation tends to induce the formation of needle-like crystals. The role of additives lies in their ability to alter the interactions between the solute and the crystal lattice, thereby influencing the development of the crystal form. Temperature control is also essential, as rapid cooling tends to produce flaky crystals, whereas slow cooling favors the formation of denser crystal structures. Increasing stirring speed generally results in smaller crystal sizes and a more uniform distribution. Other factors affecting crystal habit include smaller crystal sizes and more uniform distribution at high stirring speeds, delayed nucleation and the formation of symmetric crystals when solute-solvent interactions are strong, and the beneficial effect of ultrasonic treatment in preparing crystals with narrow particle size distribution and spherical shapes. Among these numerous influencing factors, the use of macromolecular additives plays a crucial role in the regulation of crystal habit.

Additives are primarily divided into small-molecule and macromolecule additives. Compared to small-molecule additives, macromolecular additives have the advantage of larger molecular weight and complex molecular structure, providing rich active sites that can stably adsorb to the crystal surface. According to literature, macromolecular additives can significantly affect the nucleation and growth process of crystals even at ppm concentrations. The stagnation areas of crystal growth are closely related to the geometric roughness of the crystal faces. Two-dimensional structures often form cavities or pocket-like features on crystal faces, which tend to trap solvent molecules and occupy potential solute adsorption sites, thereby inhibiting crystal growth. When macromolecular additives interact with these crystal faces, their steric hindrance effects effectively slow down the solvation of the crystal faces, allowing for precise control of the crystal growth rate. Based on this, this study focuses on protein-based macromolecular additives, represented by human serum albumin and others.

Aripiprazole (APZ), chemically known as 7-{4-[4-(2,3-dichlorophenyl)-1-piperazinylbutoxy]}-3,4-dihydro-2(1H)-quinolinone, with the chemical formula C₂₃H₂₇Cl₂N₃O₂, is primarily used as an antipsychotic drug for the treatment of various types of schizophrenia. Clinical trials abroad have shown that aripiprazole is effective in treating both the positive and negative symptoms of schizophrenia, improving associated mood symptoms, and reducing the relapse rate of schizophrenia. The chemical structure of APZ is shown below.

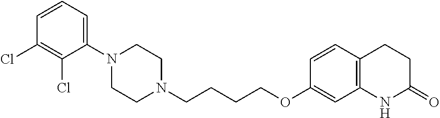 [SMILES: O=C1CCc2ccc(OCCCCN3CCN(c4cccc(Cl)c4Cl)CC3)cc2N1]

According to existing reports, APZ has 9 anhydrous crystal forms, 4 solvates, and 4 hydrate crystal forms, each crystal form corresponding to different crystal habits. For example, U.S. Pat. No. 7,910,589 disclosed the crystal forms A, B, C, D, E, G, and amorphous forms of APZ, invented by Otsuka Pharmaceutical Co., Ltd. in Japan. U.S. Pat. No. 7,504,504 discloses crystal forms I, II, VI, VIII, X, and others, disclosed by Teva Pharmaceuticals in India.

The PCT patent application WO 03/026659 discloses six anhydrous crystal forms of aripiprazole, namely B, C, D, E, F, and G. Among these, crystal form B is widely used in pharmaceutical formulations due to its low hygroscopicity. There are several methods for preparing crystal form B, such as the method disclosed in CN101948426, which involves dissolving APZ in a mixed solvent system (butanone and acetone), followed by cooling to crystallize and then filtering and drying. In Chinese patent CN114644588, a tubular continuous crystallization method for preparing crystal form B is disclosed. Chinese patent CN105924393 introduces a new crystal form of APZ, crystal form M, with an X-ray powder diffraction (XRPD) pattern showing 2θ values at 12.54, 12.70, 15.06, 17.44, 18.14, 18.76, 19.44, 20.52, 22.54, 24.84, and 35.79. The preparation of crystal form M involves the use of a large amount of organic solvents. Chinese patent CN108602774 discloses a crystal form N of APZ (which is an aripiprazole hydrate), with an XRPD pattern showing 2θ values of 12.6±0.1°, 15.1±0.1°, 17.4±0.1°, 18.2±0.1°, 18.7±0.1°, 22.5±0.1°, 23.2±0.1°, 24.8±0.1°, and 27.5±0.1°. In Chinese patent CN106674103, a preparation method for crystal form α is disclosed, with an XRPD pattern showing 2θ values at 17.4, 18.1, 19.6, 23.2, 24.4, and 27.8. The preparation of crystal form a requires conditions at −5° C.

However, most of the above methods are somewhat blind and rely solely on traditional solvent and temperature adjustments. These methods often involve the use of large amounts of organic solvents, and the reaction processes typically require low temperatures or specific temperature conditions. This not only impacts the safety of the drug but also poses challenges to the green development of the environment and the health of pharmaceutical workers.

### SUMMARY

The purpose of the present disclosure is to provide a method for obtaining two aripiprazole crystal forms, APZ-H₁ and APZ-H₂, by adding protein to the APZ crystallization system, while also discovering a method to achieve different crystal habits of a same crystal form of APZ in three different solvent systems.

The second objective of the present disclosure is to provide two new crystal forms.

To achieve this, the first technical solution provided by the present disclosure is as follows:
adding human serum albumin to first solvent, to obtain human serum albumin solution; adding the aripiprazole to second solvent, to obtain aripiprazole solution after completely dissolved; adding the aripiprazole solution to the human serum albumin solution in drops, and cooling to room temperature, to obtain the aripiprazole drug; wherein a mass ratio of the human serum albumin to the aripiprazole is 1:1 to 1:10.

Furthermore, the second solvent is one of acetonitrile, isopropanol, acetone, or methanol.

The present disclosure further provides aripiprazole crystal forms of aripiprazole drug prepared by the method for simultaneously regulating crystal forms and crystal habits of the aripiprazole drug, the aripiprazole crystal forms are APZ-H₁ and APZ-H₂.

Furthermore, 2θ values of an X-ray powder diffraction (XRPD) pattern of the APZ-H₁ are 5.74°±0.2°, 7.52°±0.2°, 11.47°±0.2°, 17.28°±0.2°, 18.71°±0.2°, 19.79°±0.2°, 22.01°±0.2°, 23.01°±0.2°, 24.32°±0.2°, 26.84°±0.2°.

Furthermore, 2θ values of an X-ray powder diffraction (XRPD) pattern of the APZ-H₂ are 7.48°±0.2°, 11.49°±0.2°, 12.37°±0.2°, 13.23°±0.2°, 15.05°±0.2°, 17.53°±0.2°, 18.65°±0.2°, 19.45°±0.2°, 20.47°±0.2°, 22.01°±0.2°, 22.58°±0.2°, 24.29°−0.2°, 24.83°±0.2°, 31.17°±0.2°.

Compared with the existing technology, the technical solution provided by the present disclosure has the following technical advantages.
(1) The technical solution provided by the present disclosure adopts protein as a regulator added to the APZ solvent crystallization system to control the crystal habit of aripiprazole drug, which results in the same crystal form but different crystal habits; additionally, two new crystal forms of aripiprazole drug were discovered during this process.
(2) The protein used in the technical solution is a substance essential for the human body, when added to the drug, it is not only non-toxic and harmless but also meets the nutritional needs of the human body. At the same time, this avoids the use of large amounts of toxic and harmful organic solvents. This solution not only regulates the crystal habit of the drug but also aligns with the national advocacy for environmentally friendly and green production.
(3) The technical solution provided by the present disclosure studies the effect of human serum albumin at different concentrations on the crystal habit of the aripiprazole drug. Experimental results show that as the protein concentration increases, the crystal habit of aripiprazole drug exhibits increasingly curved characteristics.
(4) The technical solution provided by the present disclosure is based on the interaction between human serum albumin and aripiprazole, achieving efficient control of the crystal habit, which provides guidance for the production of subsequent drug formulations.

### BRIEF DESCRIPTION OF THE DRAWINGS

FIG. 1 is a X-ray powder diffraction pattern of aripiprazole drug with crystal forms of APZ-H₁ and APZ-H₂.

FIG. 2 is a microscopic observation image of aripiprazole drug with crystal forms of APZ-H₁ obtained in the first embodiment.

FIG. 3 is a microscopic observation image of aripiprazole drug with crystal forms of APZ-H₂ obtained in the second embodiment.

FIG. 4 is a DSC (Differential Scanning calorimetry) diagram of curves of aripiprazole drug with crystal forms of APZ-H₁ and APZ-H₂.

FIG. 5 is a X-ray powder diffraction pattern of the aripiprazole drug without protein solvent in a first comparative embodiment.

FIG. 6 is a microscopic observation image of the aripiprazole drug without protein solvent in the first comparative embodiment.

FIG. 7 is the microscopic observation images of APZ under different human serum albumin concentrations.

### DETAILED DESCRIPTION OF THE EMBODIMENTS

The technical solutions in the embodiments of the present disclosure will be described clearly and completely with reference to the accompanying drawings in the embodiments of the present disclosure. Obviously, the described embodiments are only a part of the embodiments of the present disclosure rather than all of them. Based on the embodiments in the present disclosure, all other embodiments obtained by those skilled in the art without creative work shall fall within the scope of protection of the present disclosure.

### First Embodiment

The first embodiment includes the following steps:
1) dissolving 10 mg of human serum albumin (HSA) in 15 mL of deionized water to obtain an HSA aqueous solution; and
2) weighing 90 mg aripiprazole (APZ) into a conical flask, adding 15 mL methanol, and heat while stirring at 60° C. until the APZ is completely dissolved, to obtain aripiprazole solution; and
3) slowly adding the aripiprazole solution prepared in step 2) into the HSA aqueous solution prepared in step 1);
4) sealing the conical flask and making a hole in the cap, then cooling to room temperature to allow crystallization, and obtaining aripiprazole drug.

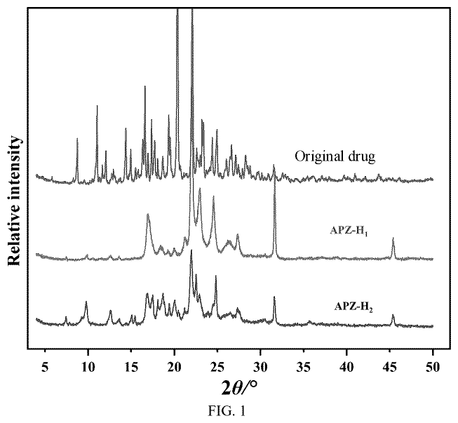

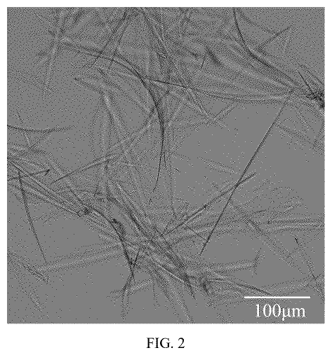

Taking a portion of the aripiprazole drug and then observing under an inverted microscope. The remaining aripiprazole drug are separated from the solution by filtration and dried at 50° C. The dried sample is then analyzed by X-ray diffraction (XRD) to obtain the corresponding crystal habit image and the XRD pattern of the sample. The crystallization of aripiprazole drug is recorded using the following X-ray powder diffraction conditions: room temperature 25° C., relative humidity<60%; X'Pert PRO polycrystalline X-ray diffractometer (PANalytical, Netherlands), Cu Kα radiation (λ=1.5406 Å), tube voltage 40 kV, tube current 40 mA, 2θ scanning range 4° to 50°, step size 0.01313°, count time 30 ms/step. The results are shown in FIG. 1. The 2θ values of aripiprazole crystal form APZ-H₁ are 5.74°, 7.52°, 11.47°, 17.28°, 18.71°, 19.79°, 22.01°, 23.01°, 24.32°, and 26.84°. The corresponding microscopic image of crystal form APZ-H₁ is shown in FIG. 2, which shows differences compared with existing crystal forms, confirming its unique crystal form and is a kind of new crystal form.

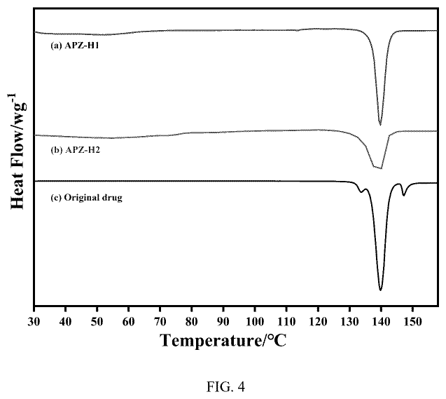

Differential scanning calorimetry (DSC) analysis is performed on the aripiprazole form APZ-H₁ prepared in the first embodiment. The differential scanning calorimetry results of the aripiprazole crystal form are recorded under the following conditions: DSC800 differential scanning calorimeter (Perkin Elmer, USA), N₂ protection, airflow rate 20 mL/min, temperature range 0° C. to 160° C., heating rate 10° C./min. The results are shown in FIG. 4. It can be seen that the DSC curve of the aripiprazole form APZ-H₁ provided in the first embodiment shows significant differences compared to the original drug, with an endothermic peak at 139.6° C., indicating a distinct difference from the original drug.

### Second Embodiment

The second embodiment includes the following steps:
1) dissolving 18 mg of HSA in 10 mL of phosphate buffer saline (PBS) to obtain an HSA buffer solution; and
2) weighing 120 mg APZ into a conical flask, adding 20 mL methanol, and heat while stirring at 60° C. until the APZ is completely dissolved, to obtain aripiprazole solution; and
3) slowly adding the aripiprazole solution prepared in step 2) into the HSA buffer solution prepared in step 1);
4) sealing the conical flask and making a hole in the cap, then cooling to room temperature to allow crystallization, and obtaining aripiprazole drug.

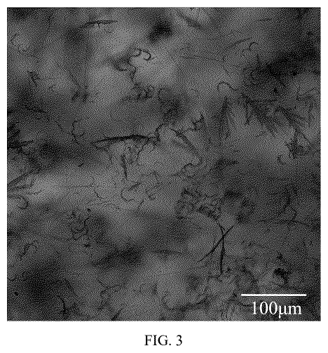

Taking a portion of the aripiprazole drug and then observing under an inverted microscope. The remaining aripiprazole drug are separated from the solution by filtration and dried at 50° C. The dried sample is then analyzed by X-ray diffraction (XRD) to obtain the corresponding crystal habit image and the XRD pattern of the sample. The crystallization of aripiprazole drug is recorded using the following X-ray powder diffraction conditions: room temperature 25° C., relative humidity<60%; X'Pert PRO polycrystalline X-ray diffractometer (PANalytical, Netherlands), Cu Kα radiation (ζ=1.5406 Å), tube voltage 40 kV, tube current 40 mA, 2θ scanning range 4° to 50°, step size 0.01313°, count time 30 ms/step. The results are shown in FIG. 1. The 2θ values of aripiprazole crystal form APZ-H₂ are 7.48°, 11.49°, 12.37°, 13.23°, 15.05°, 17.53°, 18.65°, 19.45°, 20.47°, 22.01°, 22.58°, 24.29°, 24.83° and 31.17°. The corresponding microscopic image of crystal form APZ-H₂ is shown in FIG. 3, which shows differences compared with existing crystal forms, confirming its unique crystal form and is a kind of new crystal form.

Differential scanning calorimetry (DSC) analysis is performed on the aripiprazole form APZ-H₂ prepared in the first embodiment. The differential scanning calorimetry results of the aripiprazole crystal form are recorded under the following conditions: DSC800 differential scanning calorimeter (Perkin Elmer, USA), N₂ protection, airflow rate 20 mL/min, temperature range 0° C. to 160° C., heating rate 10° C./min. The results are shown in FIG. 4. It can be seen that the DSC curve of the aripiprazole form APZ-H₂ provided in the first embodiment shows significant differences compared to the original drug, with an endothermic peak at 139.1° C., indicating a distinct difference from the original drug.

### First Comparative Embodiment

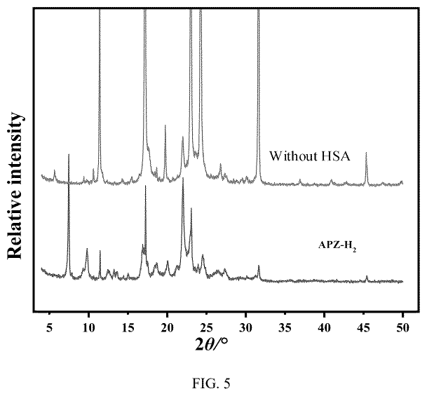

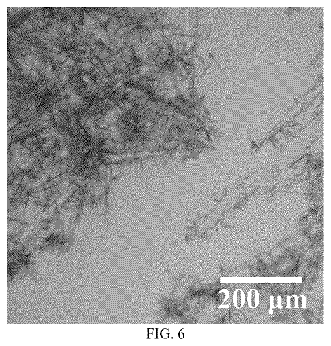

Weighing 90 mg APZ into a conical flask, adding 15 mL methanol, and heat while stirring at 60° C. until the APZ is completely dissolved, to obtain aripiprazole solution; and slowly adding the aripiprazole solution into 15 ml deionized water; sealing the conical flask and making a hole in the cap, then cooling to room temperature to allow crystallization, and obtaining aripiprazole drug. Taking a portion of the aripiprazole drug and then observing under an inverted microscope. The remaining aripiprazole drug are separated from the solution by filtration and dried at 50° C. The dried sample is then analyzed by XPRD, the final results are shown in FIG. 5. It can be observed that the aripiprazole solvent crystallization system without protein did not form any new crystal form. The crystal habit observation is shown in FIG. 6. The crystal habit of the group without protein is straight, which is significantly different from the crystal habit of the group with protein.

### Second Comparative Embodiment

Weighing 90 mg APZ into a conical flask, adding 15 mL methanol, and heat while stirring at 60° C. until the APZ is completely dissolved, to obtain aripiprazole solution; and slowly adding the aripiprazole solution into 15 ml PBS; sealing the conical flask and making a hole in the cap, then cooling to room temperature to allow crystallization, and obtaining aripiprazole drug. Taking a portion of the aripiprazole drug and then observing under an inverted microscope. The remaining aripiprazole drug are separated from the solution by filtration and dried at 50° C. The dried sample is then analyzed by XRD. The corresponding crystal habit observation and XRD pattern of the sample were obtained. The results show that, similar to the first comparative embodiment, the aripiprazole solvent crystallization system without protein did not form any new crystal form.

### Third Embodiment

The third embodiment includes the following steps:
1) dissolving 15 mg of HSA in 15 mL of deionized water to obtain an HSA aqueous solution; and
2) weighing 120 mg APZ into a conical flask, adding 20 mL methanol, and heat while stirring at 60° C. until the APZ is completely dissolved, to obtain aripiprazole solution; and
3) slowly adding the HSA aqueous solution prepared in step 1) into the aripiprazole solution prepared in step 2);
4) sealing the conical flask and making a hole in the cap, then cooling to room temperature to allow crystallization, and obtaining aripiprazole drug.

Taking a portion of the aripiprazole drug and then observing under an inverted microscope. The remaining aripiprazole drug are separated from the solution by filtration and dried at 50° C. The dried sample is then analyzed by XRD, crystal form of APZ-H₁ is obtained,

### Fourth Embodiment

The fourth embodiment includes the following steps:
1) dissolving 30 mg of HSA in 15 mL of PBS to obtain an HSA buffer solution; and
2) weighing 120 mg APZ into a conical flask, adding 25 mL methanol, and heat while stirring at 60° C. until the APZ is completely dissolved, to obtain aripiprazole solution; and
3) slowly adding the HSA buffer solution prepared in step 1) into the aripiprazole solution prepared in step 2);
4) sealing the conical flask and making a hole in the cap, then cooling to room temperature to allow crystallization, and obtaining aripiprazole drug.

Taking a portion of the aripiprazole drug and then observing under an inverted microscope. The remaining aripiprazole drug are separated from the solution by filtration and dried at 50° C. The dried sample is then analyzed by XPRD, crystal form of APZ-H₂ is obtained,

### Third Comparative Embodiment

Weighing 90 mg APZ into a conical flask, adding 15 mL methanol, and heat while stirring at 60° C. until the APZ is completely dissolved, to obtain aripiprazole solution; and slowly adding 15 ml deionized water into the aripiprazole solution; sealing the conical flask and making a hole in the cap, then cooling to room temperature to allow crystallization, and obtaining aripiprazole drug. Taking a portion of the aripiprazole drug and then observing under an inverted microscope. The remaining aripiprazole drug are separated from the solution by filtration and dried at 50° C. The dried sample is then analyzed by XPRD, The results show that, similar to the first comparative embodiment, the aripiprazole solvent crystallization system without protein did not form any new crystal form.

### Fourth Comparative Embodiment

Weighing 90 mg APZ into a conical flask, adding 15 mL methanol, and heat while stirring at 60° C. until the APZ is completely dissolved, to obtain aripiprazole solution; and slowly adding 15 ml PBS into the aripiprazole solution; sealing the conical flask and making a hole in the cap, then cooling to room temperature to allow crystallization, and obtaining aripiprazole drug. Taking a portion of the aripiprazole drug and then observing under an inverted microscope. The remaining aripiprazole drug are separated from the solution by filtration and dried at 50° C. The dried sample is then analyzed by XPRD. The corresponding crystal habit observation and XPRD pattern of the sample were obtained. The results show that, similar to the first comparative embodiment, the aripiprazole solvent crystallization system without protein did not form any new crystal form.

### Fifth Embodiment

The fifth embodiment includes the following steps:
1) dissolving 8 mg of HSA in 15 mL of deionized water to obtain an HSA aqueous solution; and
2) weighing 90 mg APZ into a conical flask, adding 20 mL acetonitrile, and heat while stirring at 60° C. until the APZ is completely dissolved, to obtain aripiprazole solution; and
3) slowly adding the aripiprazole solution prepared in step 2) into the HSA aqueous solution prepared in step 1);
4) sealing the conical flask and making a hole in the cap, then cooling to room temperature to allow crystallization, and obtaining aripiprazole drug.

Taking a portion of the aripiprazole drug and then observing under an inverted microscope. The remaining aripiprazole drug are separated from the solution by filtration and dried at 50° C. The dried sample is then analyzed by XRD, crystal form of APZ-H₁ is obtained,

### Sixth Embodiment

The sixth embodiment includes the following steps:
1) dissolving 45 mg of HSA in 15 mL of PBS to obtain an HSA buffer solution; and
2) weighing 90 mg APZ into a conical flask, adding 15 mL acetonitrile, and heat while stirring at 60° C. until the APZ is completely dissolved, to obtain aripiprazole solution; and
3) slowly adding the aripiprazole solution prepared in step 2) into the HSA buffer solution prepared in step 1);
4) sealing the conical flask and making a hole in the cap, then cooling to room temperature to allow crystallization, and obtaining aripiprazole drug.

Taking a portion of the aripiprazole drug and then observing under an inverted microscope. The remaining aripiprazole drug are separated from the solution by filtration and dried at 50° C. The dried sample is then analyzed by XPRD, crystal form of APZ-H₂ is obtained,

### Fifth Comparative Embodiment

Weighing 90 mg APZ into a conical flask, adding 15 mL acetonitrile, and heat while stirring at 60° C. until the APZ is completely dissolved, to obtain aripiprazole solution; and slowly adding the aripiprazole solution into 15 ml deionized water; sealing the conical flask and making a hole in the cap, then cooling to room temperature to allow crystallization, and obtaining aripiprazole drug Taking a portion of the aripiprazole drug and then observing under an inverted microscope. The remaining aripiprazole drug are separated from the solution by filtration and dried at 50° C. The dried sample is then analyzed by XPRD, the corresponding crystal habit observation and XRD patterns of the sample were obtained. The results show that, similar to the first comparative embodiment, the aripiprazole solvent crystallization system without protein did not form any new crystal form.

### Sixth Comparative Embodiment

Weighing 90 mg APZ into a conical flask, adding 15 mL acetonitrile, and heat while stirring at 60° C. until the APZ is completely dissolved, to obtain aripiprazole solution; and slowly adding the aripiprazole solution into 15 ml PBS; sealing the conical flask and making a hole in the cap, then cooling to room temperature to allow crystallization, and obtaining aripiprazole drug. Taking a portion of the aripiprazole drug and then observing under an inverted microscope. The remaining aripiprazole drug are separated from the solution by filtration and dried at 50° C. The dried sample is then analyzed by XPRD. The corresponding crystal habit observation and XPRD pattern of the sample were obtained. The results show that, similar to the first comparative embodiment, the aripiprazole solvent crystallization system without protein did not form any new crystal form.

### Seventh Embodiment

The seventh embodiment includes the following steps:
1) dissolving 30 mg of HSA in 15 mL of deionized water to obtain an HSA aqueous solution; and
2) weighing 90 mg APZ into a conical flask, adding 20 mL acetonitrile, and heat while stirring at 60° C. until the APZ is completely dissolved, to obtain aripiprazole solution; and
3) slowly adding the HSA buffer solution prepared in step 1) into the aripiprazole solution prepared in step 2);
4) sealing the conical flask and making a hole in the cap, then cooling to room temperature to allow crystallization, and obtaining aripiprazole drug.

Taking a portion of the aripiprazole drug and then observing under an inverted microscope. The remaining aripiprazole drug are separated from the solution by filtration and dried at 50° C. The dried sample is then analyzed by XPRD, crystal form of APZ-H₂ is obtained,

### Eighth Embodiment

The eighth embodiment includes the following steps:
1) dissolving 40 mg of HSA in 10 mL of PBS to obtain an HSA buffer solution; and
2) weighing 90 mg APZ into a conical flask, adding 15 mL acetonitrile, and heat while stirring at 60° C. until the APZ is completely dissolved, to obtain aripiprazole solution; and
3) slowly adding the HSA buffer solution prepared in step 1) into the aripiprazole solution prepared in step 2);
4) sealing the conical flask and making a hole in the cap, then cooling to room temperature to allow crystallization, and obtaining aripiprazole drug.

Taking a portion of the aripiprazole drug and then observing under an inverted microscope. The remaining aripiprazole drug are separated from the solution by filtration and dried at 50° C. The dried sample is then analyzed by XPRD, crystal form of APZ-H₂ is obtained,

### Seventh Comparative Embodiment

Weighing 90 mg APZ into a conical flask, adding 15 mL acetonitrile, and heat while stirring at 60° C. until the APZ is completely dissolved, to obtain aripiprazole solution; and slowly adding 15 ml deionized water into the aripiprazole solution; sealing the conical flask and making a hole in the cap, then cooling to room temperature to allow crystallization, and obtaining aripiprazole drug. Taking a portion of the aripiprazole drug and then observing under an inverted microscope. The remaining aripiprazole drug are separated from the solution by filtration and dried at 50° C. The dried sample is then analyzed by XPRD. The corresponding crystal habit observation and XPRD pattern of the sample were obtained. The results show that, similar to the first comparative embodiment, the aripiprazole solvent crystallization system without protein did not form any new crystal form.

### Eighth Comparative Embodiment

Weighing 90 mg APZ into a conical flask, adding 15 mL acetonitrile, and heat while stirring at 60° C. until the APZ is completely dissolved, to obtain aripiprazole solution; and slowly adding 15 ml PBS into the aripiprazole solution; sealing the conical flask and making a hole in the cap, then cooling to room temperature to allow crystallization, and obtaining aripiprazole drug. Taking a portion of the aripiprazole drug and then observing under an inverted microscope. The remaining aripiprazole drug are separated from the solution by filtration and dried at 50° C. The dried sample is then analyzed by XPRD. The corresponding crystal habit observation and XPRD pattern of the sample were obtained. The results show that, similar to the first comparative embodiment, the aripiprazole solvent crystallization system without protein did not form any new crystal form.

### Ninth Embodiment

The ninth embodiment includes the following steps:
1) dissolving 20 mg of HSA in 8 mL of deionized water to obtain an HSA aqueous solution; and
2) weighing 90 mg APZ into a conical flask, adding 15 mL acetone, and heat while stirring at 60° C. until the APZ is completely dissolved, to obtain aripiprazole solution; and
3) slowly adding the aripiprazole solution prepared in step 2) into the HSA aqueous solution prepared in step 1);
4) sealing the conical flask and making a hole in the cap, then cooling to room temperature to allow crystallization, and obtaining aripiprazole drug.

Taking a portion of the aripiprazole drug and then observing under an inverted microscope. The remaining aripiprazole drug are separated from the solution by filtration and dried at 50° C. The dried sample is then analyzed by XPRD, crystal form of APZ-H₂ is obtained,

### Tenth Embodiment

The tenth embodiment includes the following steps:
1) dissolving 28 mg of HSA in 15 mL of PBS to obtain an HSA buffer solution; and
2) weighing 90 mg APZ into a conical flask, adding 15 mL acetone, and heat while stirring at 60° C. until the APZ is completely dissolved, to obtain aripiprazole solution; and
3) slowly adding the aripiprazole solution prepared in step 2) into the HSA buffer solution prepared in step 1);
4) sealing the conical flask and making a hole in the cap, then cooling to room temperature to allow crystallization, and obtaining aripiprazole drug.

Taking a portion of the aripiprazole drug and then observing under an inverted microscope. The remaining aripiprazole drug are separated from the solution by filtration and dried at 50° C. The dried sample is then analyzed by XPRD, crystal form of APZ-H₂ is obtained,

### Ninth Comparative Embodiment

Weighing 90 mg APZ into a conical flask, adding 15 mL acetone, and heat while stirring at 60° C. until the APZ is completely dissolved, to obtain aripiprazole solution; and slowly adding the aripiprazole solution Z into 15 ml deionized water; sealing the conical flask and making a hole in the cap, then cooling to room temperature to allow crystallization, and obtaining aripiprazole drug. Taking a portion of the aripiprazole drug and then observing under an inverted microscope. The remaining aripiprazole drug are separated from the solution by filtration and dried at 50° C. The dried sample is then analyzed by XPRD, the corresponding crystal habit observation and XRD patterns of the sample were obtained. The results show that, similar to the first comparative embodiment, the aripiprazole solvent crystallization system without protein did not form any new crystal form.

### Tenth Comparative Embodiment

Weighing 90 mg APZ into a conical flask, adding 15 mL acetone, and heat while stirring at 60° C. until the APZ is completely dissolved, to obtain aripiprazole solution; and slowly adding the aripiprazole solution into 15 ml PBS; sealing the conical flask and making a hole in the cap, then cooling to room temperature to allow crystallization, and obtaining aripiprazole drug. Taking a portion of the aripiprazole drug and then observing under an inverted microscope. The remaining aripiprazole drug are separated from the solution by filtration and dried at 50° C. The dried sample is then analyzed by XPRD. The corresponding crystal habit observation and XRD pattern of the sample were obtained. The results show that, similar to the first comparative embodiment, the aripiprazole solvent crystallization system without protein did not form any new crystal form.

### Eleventh Embodiment

The eleventh embodiment includes the following steps:
1) dissolving 38 mg of HSA in 15 mL of deionized water to obtain an HSA aqueous solution; and
2) weighing 90 mg APZ into a conical flask, adding 15 mL acetone, and heat while stirring at 60° C. until the APZ is completely dissolved, to obtain aripiprazole solution; and
3) slowly adding the HSA aqueous solution prepared in step 1) in to the aripiprazole solution prepared in step 2);
4) sealing the conical flask and making a hole in the cap, then cooling to room temperature to allow crystallization, and obtaining aripiprazole drug.

Taking a portion of the aripiprazole drug and then observing under an inverted microscope. The remaining aripiprazole drug are separated from the solution by filtration and dried at 50° C. The dried sample is then analyzed by XPRD, crystal form of APZ-H₂ is obtained,

### Twelfth Embodiment

The twelfth embodiment includes the following steps:
1) dissolving 18 mg of HSA in 30 mL of PBS to obtain an HSA buffer solution; and
2) weighing 90 mg APZ into a conical flask, adding 15 mL acetone, and heat while stirring at 60° C. until the APZ is completely dissolved, to obtain aripiprazole solution; and
3) slowly adding the HSA buffer solution prepared in step 1) into the aripiprazole solution prepared in step 2);
4) sealing the conical flask and making a hole in the cap, then cooling to room temperature to allow crystallization, and obtaining aripiprazole drug.

Taking a portion of the aripiprazole drug and then observing under an inverted microscope. The remaining aripiprazole drug are separated from the solution by filtration and dried at 50° C. The dried sample is then analyzed by XPRD, crystal form of APZ-H₁ is obtained,

### Eleventh Comparative Embodiment

Weighing 90 mg APZ into a conical flask, adding 15 mL acetone, and heat while stirring at 60° C. until the APZ is completely dissolved, to obtain aripiprazole solution; and slowly adding 15 ml deionized water into the aripiprazole solution; sealing the conical flask and making a hole in the cap, then cooling to room temperature to allow crystallization, and obtaining aripiprazole drug. Taking a portion of the aripiprazole drug and then observing under an inverted microscope. The remaining aripiprazole drug are separated from the solution by filtration and dried at 50° C. The dried sample is then analyzed by XPRD, the corresponding crystal habit observation and XRD patterns of the sample were obtained. The results show that, similar to the first comparative embodiment, the aripiprazole solvent crystallization system without protein did not form any new crystal form.

### Twelfth Comparative Embodiment

Weighing 90 mg APZ into a conical flask, adding 15 mL acetone, and heat while stirring at 60° C. until the APZ is completely dissolved, to obtain aripiprazole solution; and slowly adding 15 ml PBS into the aripiprazole solution; sealing the conical flask and making a hole in the cap, then cooling to room temperature to allow crystallization, and obtaining aripiprazole drug. Taking a portion of the aripiprazole drug and then observing under an inverted microscope. The remaining aripiprazole drug are separated from the solution by filtration and dried at 50° C. The dried sample is then analyzed by XPRD. The corresponding crystal habit observation and XRD pattern of the sample were obtained. The results show that, similar to the first comparative embodiment, the aripiprazole solvent crystallization system without protein did not form any new crystal form.

### Thirteenth Embodiment

The thirteenth embodiment includes the following steps:
1) weighing 4.5 mg, 6 mg, 9 mg, 18 mg, and 45 mg of HSA separately, and dissolve each in 15 mL of buffer solution to obtain five groups of HSA buffer solutions; and
2) weighing 90 mg APZ into each of five conical flasks, adding 15 mL methanol to each conical flask, and heat while stirring at 60° C. until the APZ is completely dissolved, to obtain aripiprazole solution; and
3) slowly adding the each aripiprazole solution prepared in step 2) into each HSA buffer solution prepared in step 1);
4) sealing the conical flask and making a hole in the cap, then cooling to room temperature to allow crystallization, and obtaining aripiprazole drug.

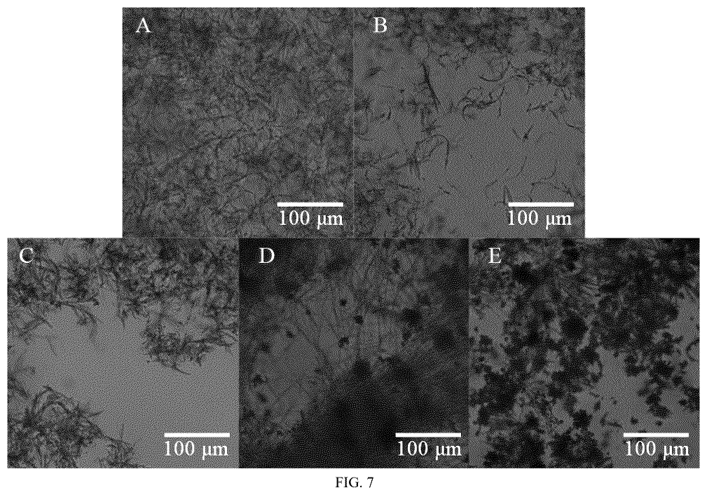

Taking a portion of the aripiprazole drug and then observing under an inverted microscope, the microscopic observation images are shown in FIG. 7, A-E correspond to different HSA weight, (A) 4.5 mg human serum protein buffer solution; and (B) 6.0 mg human serum protein buffer solution; and (C) 9.0 mg human serum protein buffer solution; and (D) 18.0 mg human serum protein buffer solution; and (E) 45.0 mg human serum protein buffer solution.

It can be observed that as the HSA concentration increases, the curvature of the crystal habit gradually becomes more pronounced.

### Fourteenth Embodiment

The fourteen embodiment includes the following steps:
1) weighing 4.5 mg, 6 mg, 9 mg, 18 mg, and 45 mg of HSA separately, and dissolve each in 15 mL of deionized water to obtain five groups of HSA aqueous solutions; and
2) weighing 90 mg APZ into each of five conical flasks, adding 15 mL methanol to each conical flask, and heat while stirring at 60° C. until the APZ is completely dissolved, to obtain aripiprazole solution; and
3) slowly adding each aripiprazole solution prepared in step 2) into each HSA aqueous solution prepared in step 1); and the corresponding concentrations are 5% HSA aqueous solution, 6.7% HSA aqueous solution, 10% HSA aqueous solution, 20% HSA aqueous solution, and 50% HSA aqueous solution.
4) sealing the conical flask and making a hole in the cap, then cooling to room temperature to allow crystallization, and obtaining aripiprazole drug.

Taking a portion of the aripiprazole drug and then observing under an inverted microscope, the microscopic observation images are shown in FIG. 7. It can be observed that as the HSA concentration increases, the curvature of the crystal habit gradually becomes more pronounced.

### Fifteenth Embodiment

The fifteen embodiment includes the following steps:
1) weighing 4.5 mg, 6 mg, 9 mg, 18 mg, and 45 mg of HSA separately, and dissolve each in 15 mL of deionized water to obtain five groups of HSA aqueous solutions; and
2) weighing 90 mg APZ into each of five conical flasks, adding 15 mL methanol to each conical flask, and heat while stirring at 60° C. until the APZ is completely dissolved, to obtain aripiprazole solution; and
3) slowly adding each HSA aqueous solution prepared in step 1) into each aripiprazole solution prepared in step 2) into; and the corresponding concentrations are 5% HSA aqueous solution, 6.7% HSA aqueous solution, 10% HSA aqueous solution, 20% HSA aqueous solution, and 50% HSA aqueous solution.
4) sealing the conical flask and making a hole in the cap, then cooling to room temperature to allow crystallization, and obtaining aripiprazole drug.

Taking a portion of the aripiprazole drug and then observing under an inverted microscope, the microscopic observation images are shown in FIG. 7. It can be observed that as the HSA concentration increases, the curvature of the crystal habit gradually becomes more pronounced.

To demonstrate the effectiveness of the two new crystal forms of aripiprazole drug, APZ-H₁ and APZ-H₂, provided in this application, the solubility of the original aripiprazole crystal form and the two crystal forms, APZ-H₁ and APZ-H₂, in a pH=1.2 hydrochloric acid solution was measured.

The procedure is as follows: After the original crystal form of the original aripiprazole drug and the two new crystal forms, APZ-H₁ and APZ-H₂, reach dissolution equilibrium, samples are taken and filtered through a 0.22 μm filter membrane. An appropriate amount of filtrate is then collected and the absorbance is measured at a wavelength of 249 nm using UV-Visible spectrophotometry (Chinese Pharmacopoeia, 2020 edition). The absorbance values are substituted into a standard curve to determine the solubility of the original aripiprazole crystal form and the two crystal forms, APZ-H₁ and APZ-H₂, as shown in Table 2.

TABLE 2
Solubility table

Solubility(mg/mL)

| Dissolving | Original crystal | | |
|---|---|---|---|
| medium | drug | APZ-H₁ | APZ-H₂ |
| pH = 1.2, | 0.0990 | 0.0681 | 0.251 |
| hydrochloric acid | | | |
| solution | | | |

The results in Table 2 show that in the pH=1.2 hydrochloric acid solution, the solubility of the APZ-H₂ is significantly higher than that of the original crystal form, while the solubility of the APZ-H₁ is similar to that of the original crystal form.

The above are only some embodiments of the present disclosure, and neither the words nor the drawings can limit the protection scope of the present disclosure. Any equivalent structural transformation made by using the contents of the specification and the drawings of the present disclosure under the overall concept of the present disclosure, or directly/indirectly applied in other related technical fields are included in the protection scope of the present disclosure.

What is claimed is:

1. A method for simultaneously regulating crystal forms and crystal habits of aripiprazole drug, wherein, comprising the following steps:
adding human serum albumin to first solvent, to obtain human serum albumin solution;
adding the aripiprazole (APZ) to second solvent, to obtain aripiprazole solution after completely dissolved;
adding the aripiprazole solution to the human serum albumin solution in drops, and cooling to room temperature, to obtain the aripiprazole drug;
wherein a mass ratio of the human serum albumin to the aripiprazole is 1:1 to 1:10.

2. The method for simultaneously regulating crystal forms and crystal habits of aripiprazole drug according to claim 1, wherein, the second solvent is one of acetonitrile, isopropanol, acetone, or methanol.

3. The method for simultaneously regulating crystal forms and crystal habits of aripiprazole drug according to claim 1, wherein, the first solvent is either deionized water or phosphate buffer saline (PBS).

4. Aripiprazole crystal forms of aripiprazole drug prepared by a method for simultaneously regulating crystal forms and crystal habits of the aripiprazole drug, wherein, the aripiprazole crystal forms are APZ-H₁ and APZ-H₂.

5. The aripiprazole crystal forms of the aripiprazole drug according to claim 3, wherein, 2θ values of an X-ray powder diffraction pattern of the APZ-H₁ are 5.74°±0.2°, 7.52°±0.2°, 11.47°±0.2°, 17.28°±0.2°, 18.71°±0.2°, 19.79°±0.2°, 22.01°+0.2°, 23.01°±0.2°, 24.32°±0.2°, 26.84°±0.2°.

1. The aripiprazole crystal forms of the aripiprazole drug according to claim 3, wherein, 2θ values of an X-ray powder diffraction pattern of the APZ-H₂ are 7.48°±0.2°, 11.49°±0.2°, 12.37°±0.2°, 13.23°±0.2°, 15.05°±0.2°, 17.53°±0.2°, 18.65°±0.2°, 19.45°±0.2°, 20.47°±0.2°, 22.01°±0.2°, 22.58°±0.2°, 24.29°±0.2°, 24.83°±0.2°, 31.17°±0.2°.
